(19) United States
(12) Patent Application Publication
(10) Pub. No.: US 2025/0345552 A1
(43) Pub. Date: Nov. 13, 2025

(54) SYSTEM, DEVICE AND METHOD FOR SOUND-BASED THERAPY

(71) Applicant: ROSINA MEHRA, New Delhi (IN)

(72) Inventor: ROSINA MEHRA, New Delhi (IN)

(21) Appl. No.: 19/203,290

(22) Filed: May 9, 2025

(30) Foreign Application Priority Data
May 9, 2024 (IN) 202311076724

(51) Int. Cl. A61M 21/00; G06F 3/16; G16H 10/60

(52) U.S. Cl. CPC A61M 21/00; G06F 3/165; G16H 10/60; A61M 2021/0027; A61M 2205/3303

(57) ABSTRACT

The present invention relates to a therapeutic system, device and method for sound-based therapy that combines music and frequency-based auditory stimuli to facilitate brainwave entrainment and inducing alterations in the state of consciousness, thereby resulting in therapeutic healing. The therapeutic system for sound-based therapy includes a wearable device (1) that is equipped with plurality of modules in order to provide therapy to the user with the help of sound. The sound-based therapy is provided by amalgamation of sound bites from both the specific frequency-based sound module (5) and classical raga-based sound module (6), thereby creating a unique audio environment and positively influencing and relaxing the user's state of mind.

### FIELD OF THE INVENTION

The present invention relates to therapeutic system, device and method for holistic wellness. More particularly, the present invention relates to a comprehensive sound therapy system, device, and method for holistic wellness.

### BACKGROUND OF THE INVENTION

Holistic wellness is a state of well-being that simultaneously approaches all aspects of an individual's life including physical, mental, emotional and social health. This includes transformation of an individual from being in an unwell state to a healthy and well state of mind via some alternative solutions. These alternative solutions includes various activities that helps in diverting the mind from a stressful state to a normal state. Holistic wellness includes but is not limited to music or sound therapy, art therapy, dance therapy, massage therapy, etc.

Sound therapy or sound healing therapy is defined as a type of therapy that uses specific instruments and music at some specialized sound frequencies to balance and heal body, mind and spirit of an individual. Sound therapy works on the principle that everything in the universe, including the body of an individual, is in a state of vibration. When the body and mind are in a state of imbalance or disease, sound is used to restore harmony and promote healing. Sound therapy works by aligning the vibrations of the body and mind with the energy of the universe.

IN201641021216 discloses about impact of Indian Classical Ragas on Panchamahabhuthas which provides specific combination ragas to use in music therapy to manage diseases and disorders relates to the imbalance of basic elements in the body which are known as tridoshas and trigunas in Ayurveda. More particularly, the Ragas that shows significant changes in the natural basic element ‘water’.

Hossein Mahboubi et al. Annals of Otology, Rhinology & Laryngology 2017, Vol. 126 (10) 681-687 https://doi.org/10.1177/0003489417725093 in their review article compared the effects of music therapy and music therapy combined with binaural beats on anxiety and pain in patients undergoing dental procedures. The study found that both interventions reduced anxiety and pain, but there was no significant difference between them.

Milena Engelke et al. PLOS Digit Health. 2023 January; 2 (1): e0000183. Published online 2023 Jan. 18. doi: 10.1371/journal.pdig.0000183 PMCID: PMC9931272PMID: 36812641 in their review article compared the effects of music therapy and music therapy combined with vibroacoustic stimulation on stress and mood in patients undergoing chemotherapy. The study found that both interventions improved stress and mood, but the combination intervention had a greater effect than music therapy alone.

These studies suggest that sound therapy techniques using multiple sound platforms may have some advantages over sound therapy techniques using single sound platforms in certain contexts.

Therefore, there is a need of a predetermined amalgamation of multiple forms of sound therapy techniques to generate a unique audio environment in order to manage various disorders.

### OBJECT OF THE INVENTION

The main object of the present invention is to provide a system for sound-based therapy.

Another object of the present invention is to provide a device having a wearable device for sound-based therapy.

Yet another object of the present invention is to provide a method for sound-based therapy.

Still another object of the present invention is to provide a predetermined amalgamation of multiple forms of sound therapy techniques that generates a unique audio environment in order to manage various disorders.

### SUMMARY OF THE INVENTION

The present invention relates to a comprehensive sound therapy system, device, and method for holistic wellness that combines music and frequency-based auditory stimuli to facilitate brainwave entrainment, thereby inducing alterations in the state of consciousness conducive to therapeutic healing.

In an embodiment, the present invention provides a system for sound-based therapy, comprising of a personal portable device accessed by a user; and a remote server connected to the personal portable device; wherein, said personal portable device is equipped with a plurality of modules and said plurality of modules include: i) a user registration module configured to register the user on the personal portable device; ii) an emotion detection module configured to detect and monitor an emotional state of the user; iii) a specific frequency based sound module configured to select a sound frequency based on the detected emotional state of the user from a set of sound frequencies stored in a database module; iv) a classical raga based sound module configured to map the emotional state of the user to one or more ragas stored in the database module; v) a specific musical instrument based module configured to select one or more instrumental frequencies stored in the database module based on the emotional state of the user; vi) a sound bite generation module configured to integrate the selected sound frequency, the one or more ragas along with the one or more instrumental frequencies to generate a sound bite that provides a customized audio environment for improving the emotional state of the user; vii) a speaker module for playing the sound bite to deliver the customized audio environment to the user, thereby providing sound-based therapy to the user; and viii) said database module is connected to the remote server for retrieving and storing a required data from the remote server.

In another embodiment, the present invention provides a method for sound-based therapy comprising steps of: a) allowing a personal portable device to be accessed by a user; b) registering, via the user registration module, the user on the personal portable device; c) detecting and monitoring, via the emotion detection module, an emotional state of the user; d) selecting, via the specific frequency based sound module, a sound frequency based on the detected emotional state of the user from a set of sound frequencies stored in a database module; e) mapping, via the classical raga based sound module, the emotional state of the user to one or more ragas stored in the database module; f) selecting, via the specific musical instrument based module, one or more instrumental frequencies stored in the database module based on the emotional state of the user; g) integrating, via the sound bite generation module, the selected sound frequency, the one or more ragas along with the one or more instrumental frequencies to generate a sound bite that provides a customized audio environment for improving the emotional state of the user; and h) playing, via the speaker module, the sound bite to deliver the customized audio environment to the user, thereby providing sound-based therapy to the user.

The above objects and advantages of the present invention will become apparent from the hereinafter set forth brief description of the drawings, detailed description of the invention, and claims appended herewith.

### BRIEF DESCRIPTION OF THE DRAWING

An understanding of the system, device and method for sound-based therapy of the present invention may be obtained by reference to the following drawing:

FIG. 1 is a block diagram of the system for sound-based therapy according to an embodiment of the present invention.

FIG. 2 is a block diagram of the wearable device for providing sound-based therapy according to an embodiment of the present invention.

FIG. 3 is a flow diagram of working of the wearable device for providing sound-based therapy according to an embodiment of the present invention.

FIG. 4 is a flow chart illustrating the creation of sound bites in the system and device, according to the present invention.

FIG. 5 is a flow chart illustrating a creation of a dataset of the combined sound bites, according to the present invention.

FIG. 6 is a flow chart illustrating an alternate method for a creation of a dataset of the combined sound bites, according to the present invention.

FIG. 7 is a graphical representation showing different types of ragas at different time intervals, according to the present invention.

### DETAILED DESCRIPTION OF THE INVENTION

The present invention will now be described hereinafter with reference to the accompanying drawings in which a preferred embodiment of the invention is shown. This invention may, however, be embodied in many different forms and should not be construed as being limited to the embodiment set forth herein. Rather, the embodiment is provided so that this disclosure will be thorough, and will fully convey the scope of the invention to those skilled in the art.

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition, the phraseology and terminology employed herein are for the purpose of description and should not be regarded as limiting.

The present invention relates to a comprehensive sound therapy system, device, and method for holistic wellness that combines music and frequency-based auditory stimuli to facilitate brainwave entrainment, thereby inducing alterations in the state of consciousness conducive to therapeutic healing.

In a preferred embodiment, the present invention provides a system for sound-based therapy, comprising of a system (100) for sound-based therapy comprising of: a personal portable device (2) accessed by a user; and a remote server (3) connected to the personal portable device (2); wherein, said personal portable device (2) is equipped with a plurality of modules and said plurality of modules include: i) a user registration module (4) configured to register the user on the personal portable device (2); ii) an emotion detection module (8) configured to detect and monitor an emotional state of the user; iii) a specific frequency based sound module (5) configured to select a sound frequency based on the detected emotional state of the user from a set of sound frequencies stored in a database module (10); iv) a classical raga based sound module (6) configured to map the emotional state of the user to one or more ragas stored in the database module (10); v) a specific musical instrument based module (7) configured to select one or more instrumental frequencies stored in the database module (10) based on the emotional state of the user; vi) a sound bite generation module (11) configured to integrate the selected sound frequency, the one or more ragas along with the one or more instrumental frequencies to generate a sound bite that provides a customized audio environment for improving the emotional state of the user; vii) a speaker module (9) for playing the sound bite to deliver the customized audio environment to the user, thereby providing sound-based therapy to the user; and viii) said database module (10) is connected to the remote server (3) for retrieving and storing a required data from the remote server (3).

Here, the personal portable device (2) include a smart phone, a special purpose device, is optionally a wearable device (1) that includes a head gear, an ear piece, a smartwatch. The plurality of modules include an imaging module for providing one or more insights of the emotional state of the user. The user registration module (4) is configured to receive a set of input details including name, age, disease, emotional stage, health insights of the user, the emotion detection module (8) include a plurality of biosensors including an electroencephalogram sensor, a galvanic skin response and a heart rate sensor for detecting a set of physiological indicators associated with the emotional state of the user. Further, the set of physiological indicators include heart rate variability, blood pressure, respiratory rate, electro dermal activity, brain activity and the set of sound frequencies include brain wave frequencies DELTA (0.1 to 4 Hz), which represent the lowest frequencies with the highest amplitude, THETA (4 to 8 Hz), Alpha (8 to 12 Hz), Beta (12 to 30 Hz), Gamma (30 to 40 Hz). In addition, one or more ragas include Indian raga sounds and western classical sounds and one or more instrumental frequencies include sounds of musical instruments. The sound bite is stored as per the time of day and season, length of sound bite ranging from 3 minutes to 6 minutes and the speaker module (9) include one or more miniature speakers for delivering audio of the sound bite directly to the user.

In another preferred embodiment, the present invention provides a method (300) for sound-based therapy comprising steps of: a) allowing a personal portable device (2) to be accessed by a user; b) registering, via the user registration module (4), the user on the personal portable device (2); c) detecting and monitoring, via the emotion detection module (8), an emotional state of the user; d) selecting, via the specific frequency based sound module (5), a sound frequency based on the detected emotional state of the user from a set of sound frequencies stored in a database module (10); e) mapping, via the classical raga based sound module (6), the emotional state of the user to one or more ragas stored in the database module (10); f) selecting, via the specific musical instrument based module (7), one or more instrumental frequencies stored in the database module (10) based on the emotional state of the user; g) integrating, via the sound bite generation module (11), the selected sound frequency, the one or more ragas along with the one or more instrumental frequencies to generate a sound bite that provides a customized audio environment for improving the emotional state of the user; and h) playing, via the speaker module (9), the sound bite to deliver the customized audio environment to the user, thereby providing sound-based therapy to the user.

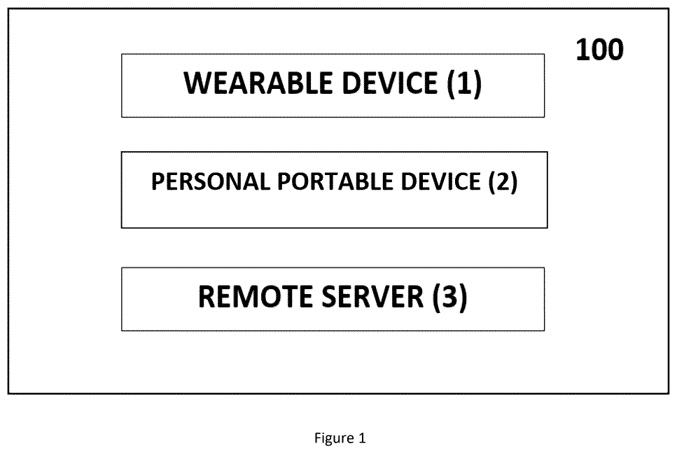

Referring to FIG. 1, a block diagram of the system for sound-based therapy is depicted. The system comprise of a wearable device (1), a personal portable device (2), and a remote server (3). The wearable device (1) is equipped with a plurality of modules that includes a user registration module (4), a specific frequency-based sound module (5), a classical raga-based sound module (6), a specific musical instrument-based module (7), a speaker module (9), an emotion detection module (8), a database module (10) for storing a plurality of music audios, frequency sounds, a sound bite generation module (11). Further the wearable device (1) include a plurality of controllers, a plurality of processors and a display unit. The wearable device (1) is worn by a user and connected to the personal portable device (2). The personal portable device (2) is accessed by the user. The user registration module (4) is configured to receive a set of input details from a user via the personal portable device (2) for registration of the user onto the wearable device (1). Following successful registration, the specific frequency-based module (5) identifies sound frequencies within predefined bandwidths stored in the database and correlates them with human emotions using the emotion detection module (8). Additionally, the classical raga-based module (6) classifies sound patterns in classical ragas and evaluates their emotional impact, aided by the emotion detection module (8). The sound bite generation module (11) amalgamates sound bites from both the specific frequency-based sound module (5) and classical raga-based sound module (6) to create a unique audio environment, fostering relaxation in the user's mind while positively influencing specific emotions under consideration.

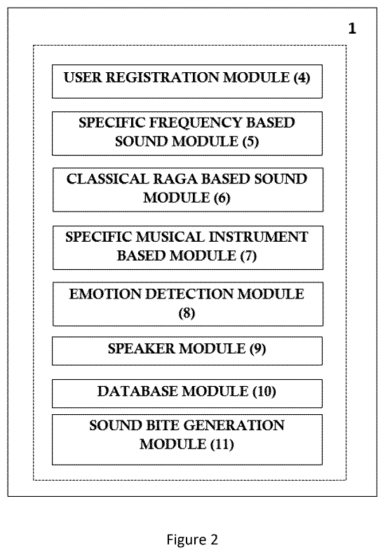

Referring to FIG. 2, a block diagram of device for sound-based therapy is depicted. The device is preferably a wearable device (1). The wearable device (1) include but not limited to head gears, eye wear or other wearable configurations. The wearable device (1) comprise of a plurality of modules, a plurality of controllers, a plurality of sensors, a remote server, a plurality of processors, frequency sounds and a display unit.

The plurality of modules are integrated within body of the wearable device (1) that include a user registration module (4), a specific frequency-based sound module (5), a classical raga-based sound module (6), a specific musical instrument-based module (7), an emotion detection module (8), a speaker module (9), a database module (10) for storing a plurality of music audios and frequency sounds, a sound bite generation module (11).

The user registration module (4) is configured to receive a set of input details from a user via the personal portable device (2) for registration of the user onto the wearable device (1). The set of input details include but not limited to name, age, gender, issue, contact details.

Following successful registration, the specific frequency-based sound module (5) identifies sound frequencies within predefined bandwidths stored in the database and correlates them with human emotions using the emotion detection module (8). The specific frequency-based sounds include brain wave frequencies DELTA (0.1 to 4 Hz), which represent the lowest frequencies with the highest amplitude, THETA (4-8 Hz), Alpha (8-12 Hz), Beta (12-30 Hz), Gamma (30-40 Hz).

Additionally, the classical raga-based sound module (6) classifies sound patterns in classical ragas and evaluates their emotional impact, aided by the emotion detection module (8).

The sound bite generation module (11) amalgamates sound bites from both the specific frequency-based sound module and classical raga-based sound module (6) to create a unique audio environment via the speaker module (9), fostering relaxation in the user's mind while positively influencing specific emotions under consideration. Further the sound bites are stored as per the time of day and season, length of sound bite, such as 3 minutes, 6 minutes and the type of instrument used in the database module (10). Further the data from the database module (10) is stored in remote server for further processing.

The plurality of controllers include an array of components, such as microprocessors, microchips, and various high-speed processors, meticulously considered to oversee a multitude of intricate operations within the wearable device (1). The plurality of controllers are not only responsible for executing and coordinating a diverse range of functions but also operate in a continuous monitoring capacity, vigilantly scrutinizing and assessing the device's performance and functionality. This constant and vigilant supervision ensures the seamless and optimal operation of the wearable device (1), guaranteeing that it consistently meets the highest standards of performance and reliability while delivering a superior user experience.

The emotion detection module (8) include plurality of sensors which further include but not limited to highly sensitive brain wave sensors, EEG sensors, temperature sensors for measuring a plurality of brain waves of the user during the therapeutic session. The sensors are adept at the precise measurement and analysis of a multitude of brainwave patterns exhibited by the user during the sound therapy sessions. By employing these sensors, the device gains invaluable insights into the user's cognitive and physiological responses, enabling it to tailor the sound therapy experience with a remarkable degree of precision and effectiveness, thereby optimizing the therapeutic benefits and user satisfaction.

The brain waves are analyzed and compared with a pre-stored data to detect the emotion at that instance of time. Further, the emotional detection module (8) analyzes the sound frequencies for determining an emotional content or emotional state associated with the sound frequency.

Furthermore, the emotion detection module (8) is a component that analyzes sound data, identifies specific frequencies within predefined ranges, and correlates them with human emotions. It does this by recognizing patterns and characteristics of sound frequencies associated with particular emotional states, such as happiness or sadness. The emotion detection module (8) uses a database module (10) and employs one or more machine learning techniques to continually improve its accuracy in detecting emotions, providing valuable insights.

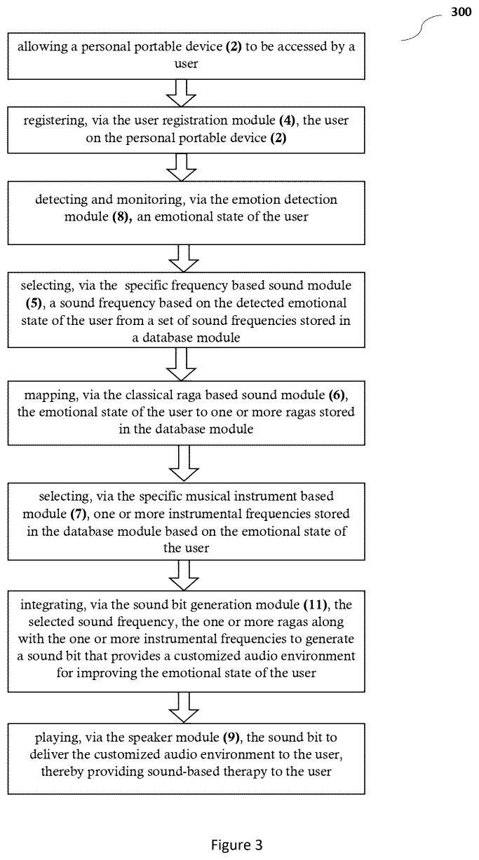

Referring to FIG. 3, is a flow chart of working of the wearable device is depicted. The wearable device (1) works according to a method that comprises a plurality of steps. At step (a), the method comprises allowing the wearable device (1) to be worn by the user. At step (b), the method comprises registering, via the user registration module (4), the user on the wearable device (1). At step (c), the method comprises detecting and monitoring, via the emotion detection module (8), an emotional state of the user and the detected emotional state is displayed onto the display unit. At step (d), the method comprises selecting, via the specific frequency based sound module (5), a sound frequency based on the detected emotional state of the user from a set of sound frequencies stored in a database module (10). At step (e), the method comprises mapping, via the classical raga based sound module (6), the emotional state of the user to one or more ragas stored in the database module (10). At step (f), the method comprises selecting, via the specific musical instrument based module (7), one or more instrumental frequencies stored in the database module (10) based on the emotional state of the user. At step (g), the method comprises integrating, via the sound bite generation module (11), the selected sound frequency, the one or more ragas along with the one or more instrumental frequencies to generate a sound bite that provides a customized audio environment for improving the emotional state of the user. At step (h), the method comprises playing, via the speaker module (9), the sound bite to deliver the customized audio environment to the user, thereby providing sound-based therapy to the user.

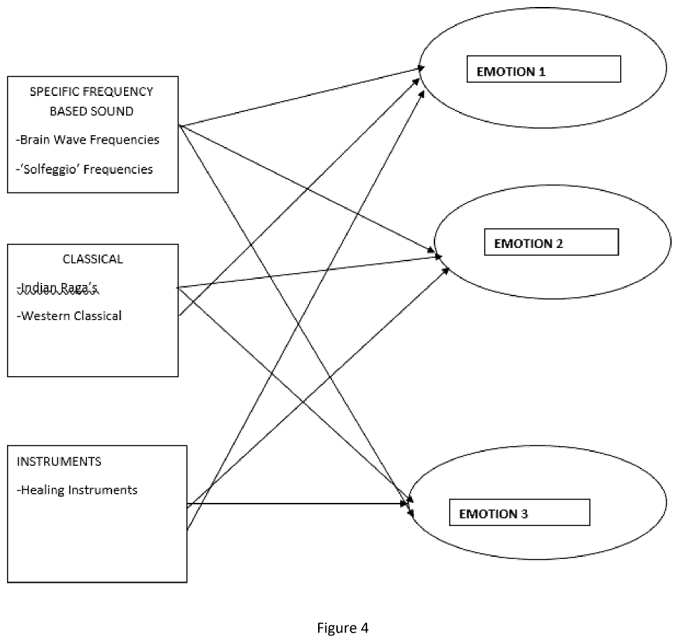

Referring to FIG. 4, is a flow chart of creation of sound bites in the system and device is depicted. The specific frequency based-sound module (5) include brain wave frequencies and solfeggio frequencies. The classical raga-based sound module (6) include Indian raga sounds and western classical sounds. The specific musical instrument based module include sounds of healing instruments.

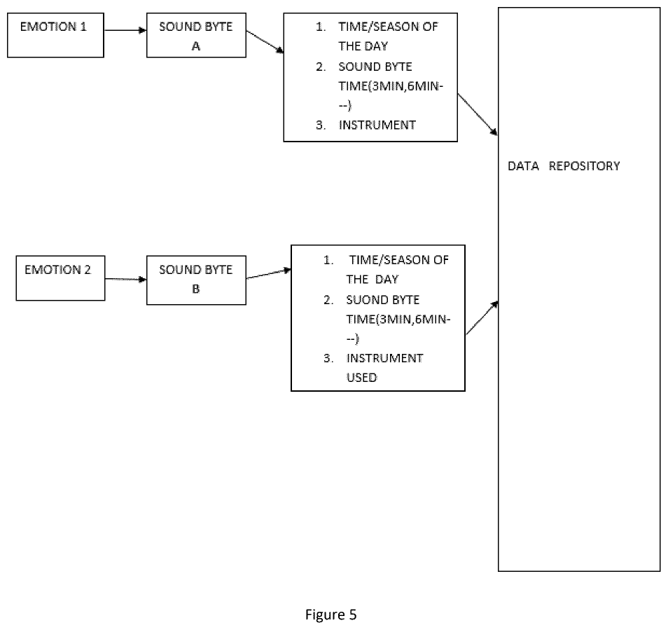

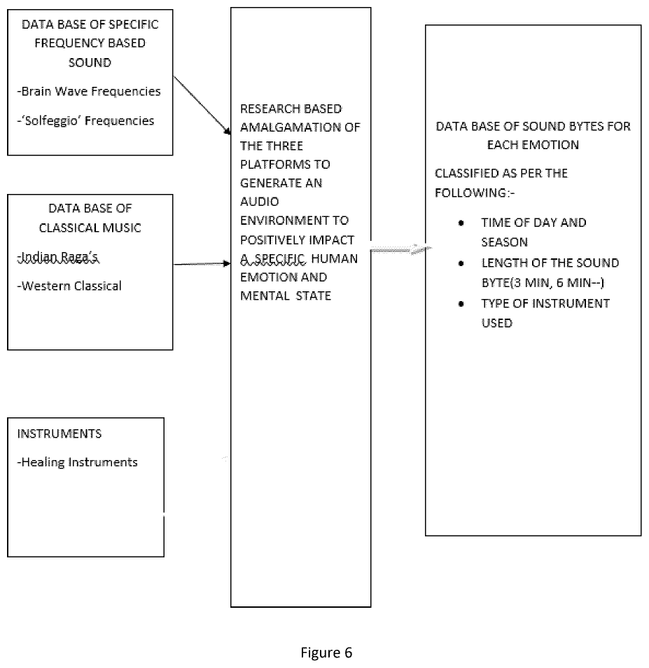

Referring to FIG. 5, is a flow chart of creating a dataset of the combined sound bites is depicted and further the sound bites are classified based on time of day/season, length of the sound bite and instrument used and an alternate method is depicted in FIG. 6.

### Example 1

### Methodology

The methodology used in the present invention is unique in that it uses a predetermined amalgamation of multiple forms of sound therapy techniques to generate a unique audio environment.

Three forms of sound based healing that are being considered in the initial stage are:—
Specific Frequency-based
Indian Classical Raga-based
Specific Musical Instrument-based
Overview of each category:
Specific Frequency-based sounds
Brain Wave Frequencies

DELTA (0.1 to 4 Hz)
Lowest Frequency with highest Amplitude;
Occurs in deep sleep;
Increase in Delta Waves decreases the awareness of the physical world;
For peak performance and high focus, then Delta Waves are reduced;
Delta waves used to induce trance, deeply relaxed states.

### THETA (4-8 Hz)

Theta waves are strong during meditation, prayer and spiritual awareness;
Theta waves represent a state between wakefulness and sleep-slow activity;
If enhanced, induces trance like state;
If suppressed, improves concentration and ability to focus.

### ALPHA (8-12 Hz)

Alpha frequencies increases by closing your eyes or deep breathing;
Alpha frequencies decreases by thinking or calculating;
When Alpha is within normal ranges—the user tend to experience good moods, see the world truthfully, and a sense of calmness;
Good healthy Alpha production induces a feeling of relaxation, in the ability to mentally coordinate and accomplish whatever task at hand;
Alpha-Theta training creates an increase in sensation, abstract thinking and self-control.

### BETA (12-30 Hz)

Beta is regarded as the normal rhythm and is dominant in those who are alert or anxious or are awake;
It is the state that most of the brain is when the user has his eyes open and are listening and thinking during analytical problem solving, judgment, decision making;
Low Beta (12-15 Hz)—produces relaxed focus and improve attentive abilities;
Mid Beta (15-18 Hz)—increases mental ability, focus and alertness;
High Beta (18-30 Hz)—enhances alertness but also produce agitation.

### GAMMA (30-40 Hz)

A good memory is associated with well regulated and efficient 40 Hz activity;
A deficiency of 40 Hz activity creates learning disabilities.
Solfeggio Frequencies
396 (Root Chakra)
Helps in feeling lighter and removes feelings of guilt, fear and grief. Basically enhances a feeling of self-confidence.
417 Hz (Sacral Chakra)
Slows down the heart rate, making it ideal for the mind to feel peaceful for Yoga, meditation and gentle exercise;
Wards off negative energy;
Cure for certain physical disorders.
528 Hz
One of the most powerful frequency—also known as the ‘Love frequency’;
Activates Intention, intuition and spiritual feeling.
639 Hz (Heart Chakra)
Binds relationships;
Eases differences within relationships.
741 Hz
Clarity of mind for a deeper understanding and also curing any chronic pains.
963 Hz
Frequency of the ‘GODS’;
Used to gain divine Consciousness;
Good for meditation.
Indian Classical Raga's

A Raga is an array of melodic structures with musical motifs, considered in the Indian tradition to have the ability to ‘color the mind’ and affect the emotions of the audience. Each Raga traditionally has an emotional significance and symbolic associations such as with season, time and mood.

Ragas with Time and Emotion:

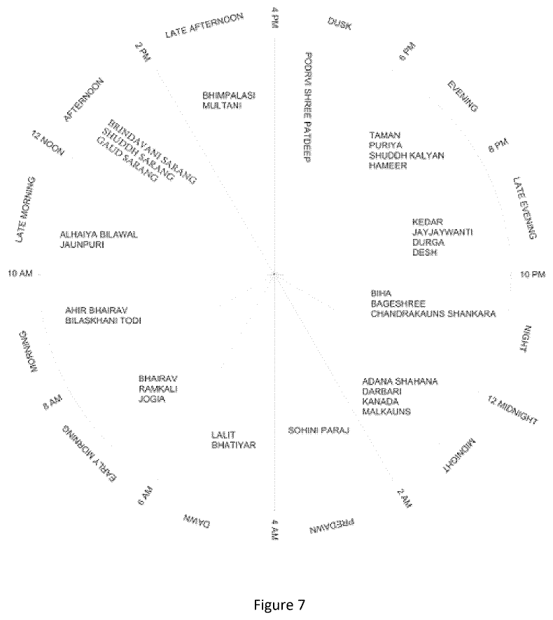

FIG. 7 shows different types of ragas according to different time intervals.

### Early Morning Raga's

Malkauns (12-2 AM): Meditative, perhaps even sad
Kanada (12-2 AM): Great emotions, feelings
Darbari (12-2 AM): Serious and solemn mood
Adana Shahana (12-2 AM): Emotions are calm and sad
Sohini Paraj (2-4 AM): Serious mood
Lalit (4-6 AM): Serene and Devotional
Bhatiyar (4-6 AM): Complete structure, awareness

### Morning Raga's:

Bhairav (6-8 AM): Solemn, peaceful
Ramkali (6-8 AM): Inspires lofty thoughts
Jogia (6-8 AM): Devotional, Detachment
Ahir Bhairav (8-10 AM): Pensiveness, Positivity, Hope
Alhaiya Bilawal (10-12 AM): Serene, Uplifting
Bilaskhani Todi (8-10 AM): Pensive, Mournful
Jaunpuri (10-12 AM): Soft, Soulful

### Afternoon Raga's:

Brindavani Sarang (12-2 PM): Summer Season, Romantic mood
Shuddh Sarang (12-2 PM): Produces a strong and lasting impression
Gaud Sarang (12-2 PM): Serious mood, hopeful, energetic atmosphere
Bhimpalasi (2-4 PM): Romantic mood, light, relaxed, calming
Multani (2-4 PM): Calm, Tranquil
Poorvi Shree Patdeep (4-6 PM): Deeply serious, quiet and mystical

### Evening and Night Raga's:

Yaman (6-8 PM): Serene, calm and peaceful
Puriya (6-8 PM): Very emotional
Shuddh Kalyan (6-8 PM): Romantic and happy
Hameer (6-8 PM): Powerful and Enthusiastic
Kedar (8-10 PM): Peacefulness
Jayjaywanti (8-10 PM): Happiness and Satisfaction
Durga (8-10 PM): Heroic and Solemn
Desh (8-10 PM): Charm
Bihag (10-12 PM): Romantic
Bageshree (10-12 AM): Emotion of Reunion
Chandrakauns Shankara (10 PM-12 AM): Anxiety and Tension

### Instruments:

Musical instrument sounds are a crucial part of the world of music, as each instrument has its unique sound and characteristics that contribute to the overall sound of a piece. The musical instrument sounds create a range of emotions and associations in the listener, from excitement and energy to intimacy and tenderness.

### Example 2

### Process Details

The present invention provides a process that is based on the well-established and scientifically proved impact of specific frequency-based sound on human emotion and similarly emotional impact of Indian ragas is also well documented. The following methodology is adopted:

Classification of each type of sound from the special frequency bandwidth and its relation with a specific Human Emotion being created by a recommended musical instrument.

Classification of each type of sound from Indian Raga's and their impact on specific Human Emotion being created by a recommended musical instrument.

This is followed by combining ‘sound bites’, affecting a similar human emotion and combining them to create a unique audio environment.

This unique audio environment provides a relaxing effect on the mind and also have a positive impact on the specific emotion being considered.

The process of combination is done in a controlled environment and under expert guidance of experts.

The system (100) for sound-based therapy comprising of a personal portable device (2) accessed by a user and a remote server (3) connected to the personal portable device (2). Further, the personal portable device (2) include a smart phone, a special purpose device, and is optionally a wearable device (1) that includes a head gear, an ear piece, a smartwatch. The plurality of modules include an imaging module for providing one or more insights of the emotional state of the user.

### Example 3

### Study Trial

### Study Sample

The study included four groups: highly-stressed group, moderately-stressed group, low-stressed group and control group. Subjects were initially recruited from a hospital and a written consent was signed by all the subjects before participation in the study by discussing with the concerned doctors. The potential subjects who were eligible to participate in the study were selected above the age of 18 years. However, the potential subjects who reported with alcohol abuse or those who had been diagnosed with a disease or those who were undergoing any treatment (specifically psychiatric treatment) were excluded from participation in the study.

### Study Design

Initially, the subjects were asked to wear the device over their head and register themselves through the personal portable device (2) by adding a set of input details-name, age, gender, contact details and issue (for which type of issue the subjects were participating in the study). The user registration module (4) is configured with the personal portable device (2) to receive the above-mentioned details from the subjects. Periodic monitoring of the emotions of the subjects was assessed via a plurality of sensors in the wearable device (1) through an emotion detection module (8). During monitoring the emotions of the subjects, some of the subjects were seem to be sweating while some were normal. There were some subjects whose heartbeat does not seem to be normal, i.e., their rate of heartbeat was increasing, some had variations in the time intervals between heartbeats while some of the subjects were having low rate of their heartbeat. It was also observed as a sign of stress that some of the subjects were shaking their legs or tapping their fingers.

Following successful registration and periodic monitoring, the frequency of stress of each subject was determined and the specific frequency-based sound module (5) identified the sound frequencies within the predefined bandwidths stored in the database and correlates them with emotions of the subjects detected by the emotion detection module (8). Thereafter, the classical raga-based sound module (6) and the specific musical instrument-based module (7) classified sound patterns in classical ragas stored in the database module (10) and evaluated their emotional impact, aided by the emotion detection module (8) as well as determined when and which sound to play. The sound bites from both the specific frequency-based sound module (5) and classical raga-based sound module (6) by the database module (10) through remove server (3) were amalgamated and played according to the frequency of stress of the respective subject through the speaker module.

### Study Measures

The brain waves of the subjects were analyzed and compared with a pre-stored data to detect the emotion at that instance of time. Further, the emotion detection module (8) determines the emotional content or emotional state associated with the respective sound frequency (Table 1).

Therefore, the present invention provides a sound-based therapeutic system, device and method for holistic wellness and also, provides a predetermined amalgamation of multiple forms of sound therapy techniques that generates a unique audio environment to manage various disorders.

Many modifications and other embodiments of the invention set forth herein will readily occur to one skilled in the art to which the invention pertain having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A system (100) for sound-based therapy comprising of:
a personal portable device (2) accessed by a user; and
a remote server (3) connected to the personal portable device (2);
wherein,
said personal portable device (2) is equipped with a plurality of modules and said plurality of modules include:
i. a user registration module (4) configured to register the user on the personal portable device (2);
ii. an emotion detection module (8) configured to detect and monitor an emotional state of the user;
iii. a specific frequency based sound module (5) configured to select a sound frequency based on the detected emotional state of the user from a set of sound frequencies stored in a database module (10);
iv. a classical raga based sound module (6) configured to map the emotional state of the user to one or more ragas stored in the database module (10);
V. a specific musical instrument based module (7) configured to select one or more instrumental frequencies stored in the database module (10) based on the emotional state of the user;
vi. a sound bite generation module (11) configured to integrate the selected sound frequency, the one or more ragas along with the one or more instrumental frequencies to generate a sound bite that provides a customized audio environment for improving the emotional state of the user;
vii. a speaker module (9) for playing the sound bite to deliver the customized audio environment to the user, thereby providing sound-based therapy to the user; and
viii. said database module (10) is connected to the remote server (3) for retrieving and storing a required data from the remote server (3).

2. The system (100) for sound-based therapy as claimed in claim 1, wherein said personal portable device (2) include a smart phone, a special purpose device.

3. The system (100) for sound-based therapy as claimed in claim 1, wherein said personal portable device (2) is optionally a wearable device (1) that includes a head gear, an ear piece, a smartwatch.

4. The system (100) for sound-based therapy as claimed in claim 1, wherein said plurality of modules include an imaging module for providing one or more insights of the emotional state of the user.

5. The system (100) for sound-based therapy as claimed in claim 1, wherein said user registration module (4) is configured to receive a set of input details including name, age, disease, emotional stage, health insights of the user.

6. The system (100) for sound-based therapy as claimed in claim 1, wherein said emotion detection module (8) include a plurality of biosensors including an electroencephalogram sensor, a galvanic skin response and a heart rate sensor for detecting a set of physiological indicators associated with the emotional state of the user.

7. The system (100) for sound-based therapy as claimed in claim 5, wherein said set of physiological indicators include heart rate variability, blood pressure, respiratory rate, electro dermal activity, brain activity.

8. The system (100) for sound-based therapy as claimed in claim 1, wherein said set of sound frequencies include brain wave frequencies DELTA (0.1 to 4 Hz), which represent the lowest frequencies with the highest amplitude, THETA (4 to 8 Hz), Alpha (8 to 12 Hz), Beta (12 to 30 Hz), Gamma (30 to 40 Hz).

9. The system (100) for sound-based therapy as claimed in claim 1, wherein said one or more ragas include Indian raga sounds and western classical sounds.

10. The system (100) for sound-based therapy as claimed in claim 1, wherein said one or more instrumental frequencies include sounds of musical instruments.

11. The system (100) for sound-based therapy as claimed in claim 1, wherein said sound bite is stored as per the time of day and season, length of sound bite ranging from 3 minutes to 6 minutes.

12. The system (100) for sound-based therapy as claimed in claim 1, wherein said speaker module (9) include one or more miniature speakers for delivering audio of the sound bite directly to the user.

13. A method (300) for sound-based therapy comprising steps of:
a) allowing a personal portable device (2) to be accessed by a user;
b) registering, via the user registration module (4), the user on the personal portable device (2);
c) detecting and monitoring, via the emotion detection module (8), an emotional state of the user;
d) selecting, via the specific frequency based sound module (5), a sound frequency based on the detected emotional state of the user from a set of sound frequencies stored in a database module (10);
e) mapping, via the classical raga based sound module (6), the emotional state of the user to one or more ragas stored in the database module (10);
f) selecting, via the specific musical instrument based module (7), one or more instrumental frequencies stored in the database module (10) based on the emotional state of the user;
g) integrating, via the sound bite generation module (11), the selected sound frequency, the one or more ragas along with the one or more instrumental frequencies to generate a sound bite that provides a customized audio environment for improving the emotional state of the user; and
h) playing, via the speaker module (9), the sound bite to deliver the customized audio environment to the user, thereby providing sound-based therapy to the user.
